(19) United States
(12) Patent Application Publication
(10) Pub. No.: US 2026/0256389 A1
(43) Pub. Date: Sep. 3, 2026

(54) REUSABLE WEARABLE UA BIOSENSING SYSTEM FOR PROLONGED MONITORING AND RISK MANAGEMENT OF HYPERURICEMIA

(71) Applicant: City University of Hong Kong, Hong Kong (HK)

(72) Inventors: Xinge YU, Hong Kong (HK); Yue HU, Hong Kong (HK); Yawen YANG, Hong Kong (HK)

(21) Appl. No.: 19/550,311

(22) Filed: Feb. 26, 2026

Related U.S. Application Data
(60) Provisional application No. 63/764,585, filed on Feb. 28, 2025.

(51) Int. Cl. A61B 5/145; A61B 5/00; A61B 5/1477

(52) U.S. Cl. CPC A61B 5/14546; A61B 5/14521; A61B 5/1477; A61B 5/6833; A61B 2562/04; A61B 2562/164

(57) ABSTRACT

The present invention discloses a wearable uric acid (UA) biosensing system comprising: a flexible printed circuit board; and a UA sensing patch attached on the flexible printed circuit board and including: an adhesive interface for on-demand removable adhesion to a skin region; an iontophoresis interface for facilitating stimulation and control of sweating of the skin region; a microfluid channel layer having a microchannel network with a plurality of microchannel inlets and a microchannel outlet for collecting sweat from the skin region; and an electrode layer including respective electrodes for sweat stimulation and UA concentration monitoring. The invention provides long-term stable and accurate uric acid monitoring through ratiometric long-life sensors resistant to by-product accumulation, a biocompatible and temperature-responsive hydrogel interface for comfortable wear, and integrated sweat induction and microfluidic collection, enabling reliable noninvasive hyperuricemia risk assessment and health management.

### CROSS-REFERENCE TO RELATED APPLICATIONS

The present application claims priority from the U.S. Provisional Patent Application No. 63/764,585 filed Feb. 28, 2025, and the disclosure of which is incorporated herein by reference in its entirety.

### FIELD OF THE INVENTION

The present invention generally relates to wearable bioelectronics. More specifically the present invention relates to reusable interference-resistant wearable bioelectronics for prolonged monitoring and risk management of hyperuricemia.

### BACKGROUND OF THE INVENTION

Hyperuricemia (HUA) has emerged as an important metabolic disorder and is currently ranked after hypertension, hyperglycemia, and hyperlipidemia in terms of prevalence. Epidemiological data indicate that HUA affects more than one billion individuals worldwide, with incidence rates continuing to rise and showing a clear trend toward younger patient populations. Clinical and epidemiological studies further indicate that HUA is frequently associated with an increased risk of comorbid conditions, including hypertension, diabetes mellitus, cardiovascular diseases, cerebrovascular disorders, as well as progressive renal impairment that may advance to kidney failure or uremia. Consequently, effective monitoring of uric acid levels is of substantial importance for early diagnosis, disease management, and long-term health risk assessment.

At present, serum uric acid measurement remains the standard clinical approach for monitoring uric acid levels. This approach relies on invasive blood sampling, which introduces discomfort, increases the potential risk of infection, and often leads to reduced patient compliance, particularly in scenarios requiring frequent or continuous monitoring. In response to these limitations, analysis of uric acid in sweat using wearable sensing devices has been proposed as a non-invasive alternative to conventional serum-based testing. Sweat-based monitoring offers the potential for continuous, real-time assessment without the need for repeated blood collection.

However, existing wearable sweat sensing technologies remain limited in their ability to support long-term and reliable uric acid monitoring. Current technical challenges include limited sensor reusability, susceptibility to chemical or biological interference, degradation of sensor performance over extended use, instability at the device-skin interface, and inefficiencies in sweat collection and transport. In particular, the lack of durable, interference-resistant sensors and integrated microfluidic systems capable of consistent sweat acquisition continues to hinder practical deployment of wearable uric acid monitoring devices in clinical and daily-life settings. These limitations highlight the need for improved wearable sensing solutions that address long-term stability, reliability, and user comfort.

### SUMMARY OF THE INVENTION

The present invention provides a wearable UA sensing system configured to support long-term, autonomous sweat collection and real-time evaluation of uric acid levels for continuous health management and timely intervention.

In accordance with one aspect of the present invention, a reusable wearable uric acid (UA) biosensing system is provided. The system comprises: a flexible printed circuit board; and a UA sensing patch attached on the flexible printed circuit board and including: an adhesive interface for on-demand removable adhesion to a skin region; an iontophoresis interface for facilitating stimulation and control of sweating of the skin region; a microfluid channel layer having a microchannel network with a plurality of microchannel inlets and a microchannel outlet for collecting sweat from the skin region; and an electrode layer including respective electrodes for sweat stimulation and UA concentration monitoring.

In one embodiment, the electrode layer includes UA sensing electrodes for measuring UA concentration in the sweat of the skin region and the UA sensing electrodes having a metal/polymer functionalized electrode structure and configured to be operated under a ratiometric sensing mode.

In one embodiment, the metal/polymer functionalized electrode structure is constructed with a metallic thin film deposited with nano metallic particles and coated with a layer of electroactive polymer.

In one embodiment, the metallic thin film and the nano metallic particles are made of gold and the electroactive polymer is a polythionine.

In one embodiment, the UA sensing electrodes are arranged in a three-electrode configuration including a working electrode (WE), a counter electrode (CE) and a reference electrode (RE) for providing a stable potential against which the potential of the working electrode is measured.

In one embodiment, the electrode layer further includes a pair of sweat induction electrodes for applying stimulation current on the skin region to induce sweating through iontophoresis effect.

In one embodiment, the iontophoresis interface is patterned to match with the sweat induction electrodes and positioned to form an iontophoresis medium between the skin and the sweat induction electrodes.

In one embodiment, the iontophoresis interface includes a cathode interfacing medium having a shape matched with the cathode of the sweat induction electrodes; and a pair of anode interfacing mediums each having a shape matched with each anode pad of the sweat induction electrodes.

In one embodiment, the cathode interfacing medium is made of a NaCl hydrogel; and the anode interfacing mediums is made of a carbachol hydrogel.

In one embodiment, the electrode layer further includes a pair of admittance analysis electrodes arranged at opposite side walls of a microchannel outlet of the microchannel network respectively such that the admittance analysis electrodes and the sweat flowing through the microchannel outlet form an electrode-electrolyte interface for quantifying sweating of the skin region.

In one embodiment, the plurality of microchannel inlets distributed over a central area of the skin region; and the microchannel outlet positioned in a peripheral area of the skin region.

In one embodiment, the plurality of microchannel inlets is evenly arranged around two opposite sides of an anode of the sweat induction electrodes in a wrap-around manner.

In one embodiment, the adhesive interface is patterned to have respective openings matched with shapes of iontophoresis interface and the plurality of microchannel inlets.

In one embodiment, the respective openings include a first opening having a shape matched with the cathode interfacing medium; a pair of second openings having shapes matched with the anode interfacing mediums; and a plurality of third openings having shapes matched with the microchannel inlets.

In one embodiment, the adhesive interface is made of a temperature-responsive adhesive hydrogel which forms a soft conformal contact with the skin and adapt with a variety of mechanical deformations.

In one embodiment, the temperature responsive adhesive hydrogel is a PCPG hydrogel.

In one embodiment, the channel layer is made of a layer of polydimethylsiloxane (PDMS) patterned to form the microchannel network.

In one embodiment, the microchannel network has a plurality of microchannels, each sprayed with a layer of monodisperesed silica (SiO₂) micropheres to improve surface hydrophilicity.

The present invention provides long-term stable and accurate uric acid monitoring through ratiometric long-life sensors resistant to by-product accumulation, a biocompatible and temperature-responsive hydrogel interface for comfortable wear, and integrated sweat induction and microfluidic collection, enabling reliable noninvasive hyperuricemia risk assessment and health management.

The wearable UA sensing system is configured for intelligent sweat sampling and uric acid monitoring under daily-life conditions up to 15 days. Evaluation results obtained from both healthy individuals and hyperuricemia populations demonstrate the applicability of the platform for non-invasive hyperuricemia risk indication, dietary management support, and medication usage guidance. Collectively, the disclosed system provides a practical and scalable solution for long-term, non-invasive uric acid monitoring using wearable technology.

### BRIEF DESCRIPTION OF THE DRAWINGS

Embodiments of the invention are described in more details hereinafter with reference to the drawings, in which:

FIG. 1 shows a wearable biosensing system (denoted as PUAMS) for prolonged uric acid (UA) monitoring and management and its implementation scenario in accordance with one embodiment of the present invention.

FIG. 2 shows a block diagram of electronic circuits of the wearable biosensing system.

FIG. 3 shows an exploded view of the wearable biosensing system.

FIG. 4 shows an exploded view of a biosensing patch in accordance with one embodiment of the present invention.

FIGS. 5A to 5D show layouts of adhesive interface, iontophoresis interface, microfluid channel layer and electrode layer of the biosensing patch respectively.

FIG. 6 shows a fabrication process and sensing principle of the ratiometric sensor for UA detection using pTh to resist by-product deposition and weaken multi-dimensional disturbances.

FIG. 7 shows a schematic diagram of the admittance analysis electrodes at the microfluidic outlet.

FIG. 8 shows an equivalent circuit model describing the conduction mechanism for the electrode-electrolyte interface formed with the admittance analysis electrodes and the microfluidic outlet.

FIG. 9 shows the body temperature-triggered gentle adhesion and ice-cooling-induced painless detachment of the PCPG hydrogel.

FIG. 10 shows an exploded view of a flexible circuit board assembly in accordance with one embodiment of the present invention.

FIG. 11A shows repetitive DPV signals of the same Nano Au electrode without pTh modification continuously in response to a 40 μM UA sample without any cleaning operations. The experiment was repeated 3 times independently with similar results; FIG. 11B shows continuous and repetitive DPV signals obtained from the fabricated ratiometric sensor (with pTh modification) in response to a 40 μM UA sample without any extra operations. The inset is the calculated ratio signals versus number of test; FIG. 11C shows alternating DPV signals of the sensors in response to UA samples of low and high concentrations, where the odd number test corresponds to the low concentration sample (10 μM) and the even number test corresponds to the high concentration sample (40 μM).

FIGS. 12A to 12C show the dependence of the sensor responses on varied pH, temperature and concentrations of ionic strength, respectively.

FIGS. 13A and 13B show dot plots of the current signal of UA alone (I_{UA}) and the ratio current signal of UA and pTh (I_{UA}/I_{pTh}) employing sensors with different working areas to detect UA samples in varied concentrations.

FIG. 14 shows long-term reliability results of sensors after being activated, their corresponding current densities of UA, pTh, and ratio signal versus storage time. All error bars represent the standard deviation from three parallel tests.

FIG. 15 shows plots of the corresponding current density versus time (1 to 18 min) when the high concentration UA sample (40 μM) is pumped into the microchannel network filled with low concentration sample (10 μM). A fixed flow rate of 1.5 μL min⁻¹ was used and DPV scans were performed every minute. The scan range of DPV was set as 0.05 to 0.4 V.

FIG. 16 shows the DPV voltammograms of the fabricated sensor for 40 μM UA sensing at different flow rates (from 0.5 to 3 μL min⁻¹).

FIG. 17 shows calibration curve between electrode admittance and electrolyte concentrations.

FIG. 18 shows study on the relationship between electrode admittance and whether the electrolyte flows or not at the outlet of microchannel. The point where the arrow points is the split point between the flow and stationary sweat. The sweat in front of the arrows in the figure flows at 1 μL min⁻¹ while the sweat behind the arrows remains stationary.

FIG. 19A shows numerically simulated UA concentration distributions; and FIG. 19B shows on-body evaluation of the optimized flexible microfluidic reservoir for carbachol-based iontophoretic sweat secreting and induction at rest. 1% carbachol and 500 μA stimulated current were employed for on-body test. Ink was injected into the reservoir in advance to facilitate visualization of sweat flow in the microfluidics.

FIG. 20 shows adhesive strengths of the PCPG hydrogel to pigskin upon heating the hydrogel to 37° C. or cooling to 10° C.

FIG. 21 shows photographs of PCPG hydrogel adhering to human skin and its allergy test. Scale bar, 2 cm.

FIG. 22 shows performance comparison of PCPG hydrogel and medical adhesives as long-term wearable device-skin interface.

FIGS. 23A and 23B show sweat and serum UA levels of subjects under regular diet and purine-rich diet over 3 days, respectively.

FIG. 24 shows comparison of UA levels in sweat and serum of gout patients, subjects with hyperuricemia, and healthy individuals. Dots represent the UA values from all the individuals in each category (n=2 for patients with gout, n=3 for subjects with hyperuricemia and n=5 for healthy subjects).

FIG. 25 shows correlation of serum and sweat UA concentrations from n=80 biologically independent human samples.

FIG. 26 shows close associations between metabolic nucleotides and nucleoproteins throughout the day and the risk level of HUA.

FIGS. 27A and 27B show scatterplot reflecting the relationship between age, BMI and uric acid concentrations in serum from 50 randomized patients with HUA, respectively. The highlighted areas inside are the age and BMI ranges for most patients, respectively.

FIG. 28 shows recorded UA fluctuations in healthy individuals of different ages and genders on a random day.

FIG. 29A shows comparison of the concentration fluctuation of sweat UA between vegetarian (male, 30 years old) and meat (male, 29 years old) lovers of subjects with similar BMI; FIG. 29B shows UA levels recorded in sweat of sugary (male, 27 years old) and non-sugary (male, 31 years old) beverage enthusiasts, respectively; and FIG. 29C shows UA levels recorded in sweat of beer (male, 50 years old) and non-beer (male, 51 years old) enthusiasts, respectively.

FIG. 30 shows sweat UA levels consecutively recorded in three individuals over a week. Healthy subject 1, male, 27 years old. Healthy subject 2, female, 25 years old. Patient with HUA, male, 32 years old. For subject 1, high-purine diet such as hot pot, beer, and seafood were consumed on the third and ninth days, respectively. For subject 2, there was no special intake of high-purine diet during the entire test period. For patient with HUA, febuxostat (a uric acid-lowering drug) was taken from the third day. ΔC(M), ΔC(H) represent the increase in the UA concentration at this point to a medium risk level and a high risk level, respectively.

### DETAILED DESCRIPTION

In the following description, details of the present invention are set forth as preferred embodiments. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

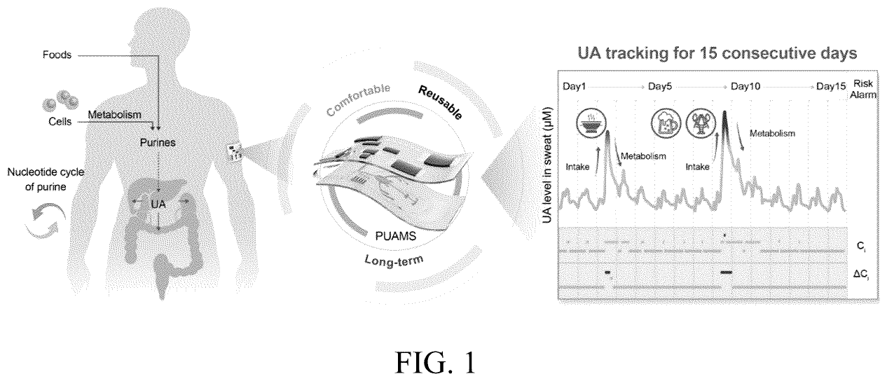

FIG. 1 illustrates a wearable biosensing system (denoted as PUAMS) for prolonged uric acid (UA) monitoring and management and its implementation scenario in accordance with one embodiment of the present invention. The wearable biosensing system enables prolonged and autonomous sweat sample collection and accurate UA evaluation at any given time with an on-site signal processing and wireless communication way.

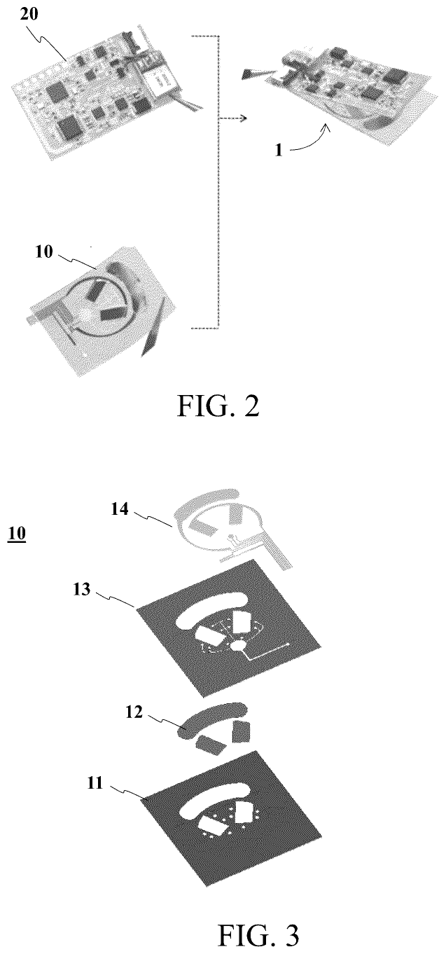

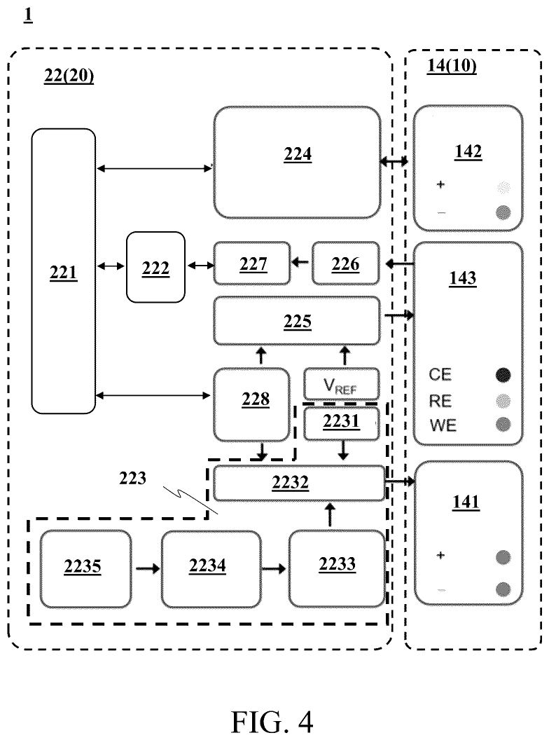

Referring to FIGS. 2 and 4, the wearable biosensing system 1 comprises a wearable sensing patch 10 and a flexible printed circuit board (FPCB) assembly 20.

Referring to FIG. 3, the wearable sensing patch 10 comprises: an adhesive interface 11 for on-demand removable adhesion to a skin region; an iontophoresis interface 12 for facilitating stimulation and control of sweating of the skin region; a microfluid channel layer 13 having a microchannel network for collecting sweat from the skin region; and an electrode layer 14 including respective electrodes for sweat stimulation and UA concentration monitoring.

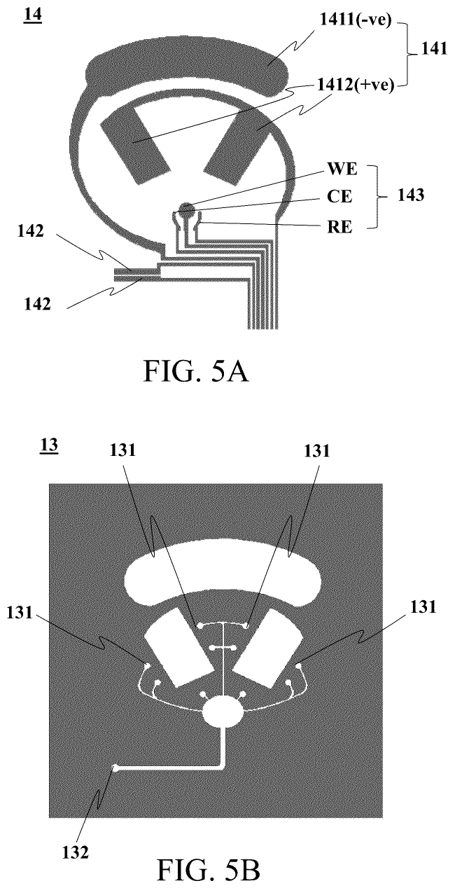

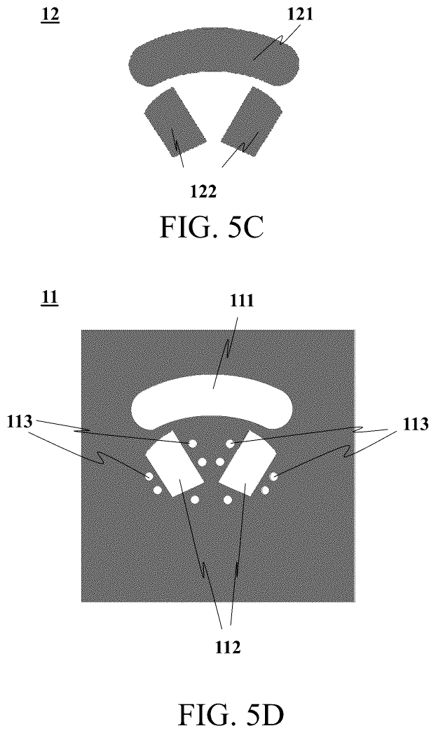

Referring to FIG. 5A, the electrode layer 14 includes: sweat induction electrodes 141 for applying stimulation current on the skin region to induce sweating through iontophoresis; admittance analysis electrodes 142 for quantifying the induced sweating of the skin region; and UA sensing electrodes 143 for measuring UA concentration in the sweat of the skin region.

In some embodiments, the electrode layer 14 is fabricated by photolithographic patterning of an electrically conductive materials (e.g. gold, silver, copper) layer disposed on a polyimide (PI) substrate.

The UA sensing electrodes 143 are configured as a three-electrode system includes a working electrode (WE), a counter electrode (CE) and a reference electrode (RE) for providing a stable potential against which the potential of the working electrode is measured.

The UA sensing electrodes have an entirely metal/polymer functionalized electrode structure and are capable of working in a ratiometric sensing mode, so that the sensing signals are resistant to interference from by-product contamination. Specifically, the UA sensing electrode was fabricated as follows: a PI/Au thin-film electrode was prepared by depositing gold onto a PI membrane substrate via gold sputtering; the obtained Au electrode was then immersed in a chloroauric acid solution to deposit gold nanoparticles, forming a nano Au electrode; finally, an electroactive polymer film was formed on the nano Au electrode surface through electropolymerization. In other words, an electroactive polymer coating was modified onto the surface of the nano Au electrode by electropolymerization. Preferably, the electroactive polymer is a polythionine (pTh).

Specifically, the three-electrode system for UA sensing and iontophoretic electrodes were fabricated on a PI film with a thickness of 75 m as a supporting substrate. First of all, the PI film was sequentially cleaned with deionized water (DI water), ethanol, and acetone. A layer of Cr/Au (10 nm/100 nm) was coated using a DENTON Explorer-22 e-Beam Evaporation System. To obtain the custom pattern, a PR layer was spin-coated on the PI/Cr/Au membrane. The optimized parameters were 500 rpm for 10 s, 3000 rpm for 30 s, and baked at 110° C. for 5 min. Then it was exposed to ultraviolet light for 45 s with the help of custom mask and a URE-2000 mask aligner model to operate photolithography. After treating with ultraviolet light, it was developed for 1 min in AZ 400K solution (Vdeveloper:Vwater was set as 1:3). Finally, Au and Cr were etched accordingly, followed by acetone was used to remove the residual PR.

For the working electrode, electrochemical preparation of gold nanoparticles (nano Au) and thionine (Thi) on the Au electrode was conducted with electrochemical method in turn. Briefly, nano Au was electrodeposited on the surface of Au electrode in 0.5 M H₂SO₄ containing 10 mM HAuCl₄ with applied potential of 0.15 V for 30 s. Subsequently, surface of the nano Au electrode was polymerized by CV scanning from −0.4 V to 0.4 V with the scan rate of 0.1 V s¹ for 30 consecutive cycles in 5 mM thionine solution which was adjusted to pH 6.0. To prepare the reference electrode, Ag/AgCl ink was screen-printed onto another piece of Au film and dried overnight.

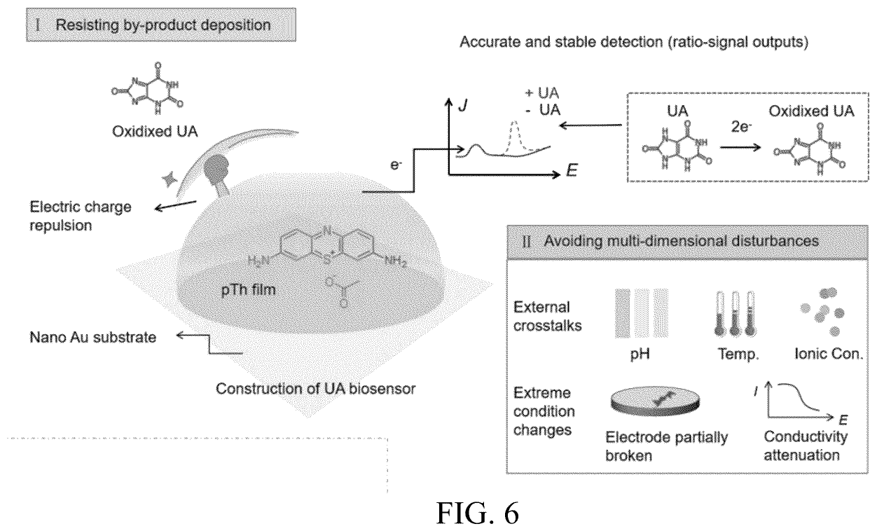

The nano Au particles can provide larger surface working area and better catalytic activity. The pTh coating can avoid the adsorption of allantoin during the UA oxidation process by virtue of the repulsive effect of the same charge, achieving prolonged re-usability (FIG. 6).

In addition, the pTh coating is facile to provide stable electrical signal output which serves as a built-in calibration signal for constructing the ratiometric sensing mode. In other words, the pTh coating layer acts as a functional material layer providing a calibration signal. With the built-in calibration signal, the sensor responses can be calibrated against variable factors (pH, temperature and ion concentration of the detection sample, electrode damage and conductivity degradation due to long term usage).

The channel layer 13 may be made of polydimethylsiloxane (PDMS) and patterned to form the microchannel network. Inner surface of each microchannel is sprayed with a layer of monodisperesed silica (SiO₂) micropheres to improve the surface hydrophilicity. Referring to FIG. 5B, in one embodiment, the microchannel network may have a plurality of microchannel inlets 131 distributed over central area of the skin region and a microchannel outlet 132 positioned in a peripheral area of the skin region.

Preferably, the plurality of microchannel inlets is arranged around the anode of the sweat induction electrodes in a wrap-around manner, which is more conducive to efficient sampling of localized perspiration than arranging the inlets on only one side of the anode. In other words, the plurality of microchannel inlets is evenly arranged on two opposite sides of the anode of the sweat induction electrodes to achieve more efficient collection of sweat.

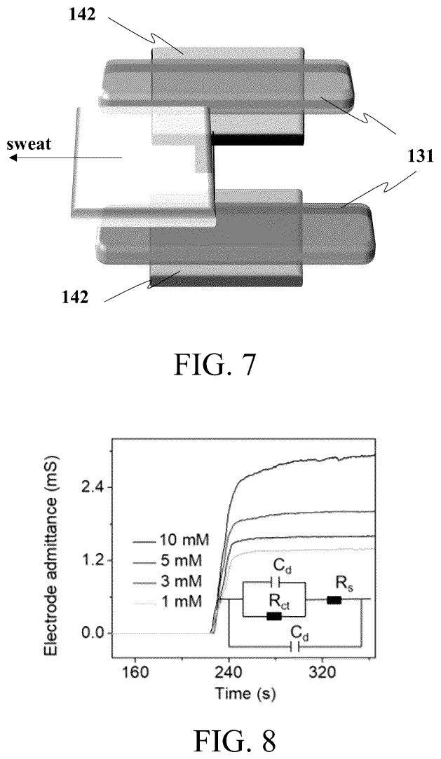

Referring to FIG. 7, the admittance analysis electrodes 142 include a pair of anode and cathode arranged at opposite side walls of the microchannel outlet 132 respectively such that the admittance analysis electrodes and the sweat flowing through the outlet 132 form an electrode-electrolyte interface (or electrode-sweat microcell). FIG. 8 illustrates an equivalent circuit model describing the conduction mechanism for the electrode-sweat microcell, where C_{d}, R_{ct}, and Rₛ represent the capacitance between the two admittance analysis electrodes, the charge-transfer resistance between the electrode and sweat, and sweat resistance, respectively. The equivalent impedance of the electrode-sweat microcell is given by

$${Z}_{sweat} ( ω ) = {R}_{s} + {( \frac{1}{{R}_{ct}} + j ω {C}_{d} )}^{- 1} .$$

So the admittance of the electrode-sweat interface can be expressed as

$${Y}_{sweat} ( ω ) = {[ {R}_{s} + {( \frac{1}{{R}_{ct}} + j ω {C}_{d} )}^{- 1} ]}^{- 1} .$$

Under high-frequency (100 kHz) and small-signal excitation the impedance of the electrode-sweat microcell is much smaller than the sweat resistance:

$${( \frac{1}{{R}_{ct}} + j ω {C}_{d} )}^{- 1} ≪ {R}_{s} .$$

Therefore, the admittance of the electrode-sweat interface is dominated by the sweat resistance and can be approximated as

$${Y}_{sweat} \approx \frac{1}{{R}_{s}} .$$

Referring to FIG. 5C, the iontophoresis interface 12 is patterned to match with the sweat induction electrodes 141 and positioned to form an iontophoresis medium between the skin and the sweat induction electrodes 141. Specifically, the iontophoresis interface 12 includes a cathode interfacing medium 121 having a shape matched with the cathode 1411 of the sweat induction electrodes 141; and a pair of anode interfacing mediums 122 each having a shape matched with each anode pad 1412 of the sweat induction electrodes 141. The cathode interfacing medium 121 may be made of a NaCl hydrogel. The anode interfacing mediums 122 may be made of a carbachol hydrogel. Under ionophoresis, a stimulation circuit is formed and perspiration is induced near the anode interfacing mediums 122.

The adhesive interface 11 is patterned to have respective openings matched with shapes of the iontophoresis interface 12 and the microchannel inlets 131. Specifically, the openings include a first opening 111 having a shape matched with the cathode interfacing medium 121; a pair of second openings 112 having shapes matched with the anode interfacing mediums 122; and a plurality of third openings 113 having shapes matched with the microchannel inlets 131.

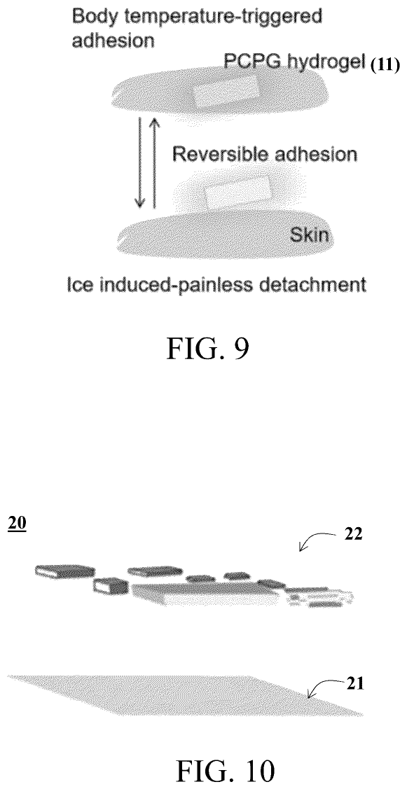

The adhesive interface 11 may be made of a temperature-responsive adhesive hydrogel which forms a soft conformal contact with the skin and adapt with a variety of mechanical deformations. In one embodiment, the temperature responsive adhesive hydrogel is a PCPG hydrogel. The PCPG hydrogel can be temperature-triggered for reversible and painless adhesion and detachment, in response to long-term wearable demand of the sensor patch (FIG. 9).

To achieve long-term reusability and wearability, the PCPG hydrogel is prepared with an optimized facile two-step strategy. Firstly, 1.08 g polyvinyl alcohol (PVA), 0.12 g gelatin, and 0.15 g chitosan, are mixed to form a mixture (i.e., PVA, gelatin and chitosan are mixed in a mass ratio of 36:4:5). Then, 3 g glycerol-water (in a mass ratio of 1:4) binary solvent and 6 mL PA aqueous solution are added to the mixture in turn with stirring to obtain a suspension liquid. The suspension liquid is then water bath heated at 80° C. until it became a clear liquid. Subsequently, the clear liquid is poured into a mold of a target shape of the adhesive layer, cooled down to room temperature and then transferred to −20° C. for 20 hours for internal hydrogen bond formation.

Referring to FIG. 10, the FPCB assembly 20 includes a flexible substrate 21; and electronic circuits 22 built on the flexible substrate. In some embodiments, the electronic circuits may have components vertically oriented and/or stacked on the flexible substrate to minimize contact area to skin for more conducive to long-term senseless wear.

Referring back to FIG. 4, the electronic circuits on the electronic circuits 22 may include a microcontroller 221 configured to control the stimulation current based on the amount of sweating and a wireless communication (e.g. Bluetooth) module 222 communicable with the microcontroller 221 and configured to receive command signals from and/or transmit the UA sensing signals to external processors.

For sweat induction, a programmable constant current source 223 is connected to the sweat induction electrodes 141 and an impedance measurement module 224 is connected to the admittance analysis electrodes 142. The current source 223 consists of an op-amp 2231 and a current mirror 2232, a high voltage booster 2233, a regulator 2234 and a power supply 2235. The impedance measurement module 224 is configured to monitor sweat collection and send data to the microcontroller 221 to shut down the current source 223 when sufficient sweat is detected.

For UA sensing, a potentiostat interface 225 is connected to the working electrode and the reference electrode of the UA sensing electrodes 143 to control the voltage (potential) between the working electrode and the reference electrode. The sensor current acquired by the UA sensing electrodes 143 is converted to voltage signals through a transimpedance amplifier (TIA) 226. The voltage signals are amplified by an instrumentation amplifier (In-Amp) 227 and then transmitted to the wireless communication module 222.

The microcontroller 221 control the current source 223 and the potentiostat interface 225 via a dual digital-to-analog converter 228 through I2C.

In one exemplary implementation, the wearable biosensing system is designed to be reusable and fabricated a thin and lightweight wearable biosensing patch with a total weight of 2.73 g that can be tightly adhered to skin. The overall dimension of the biosensing system is 4.4 cm in length, 3.3 cm in width and 4 mm in thickness.

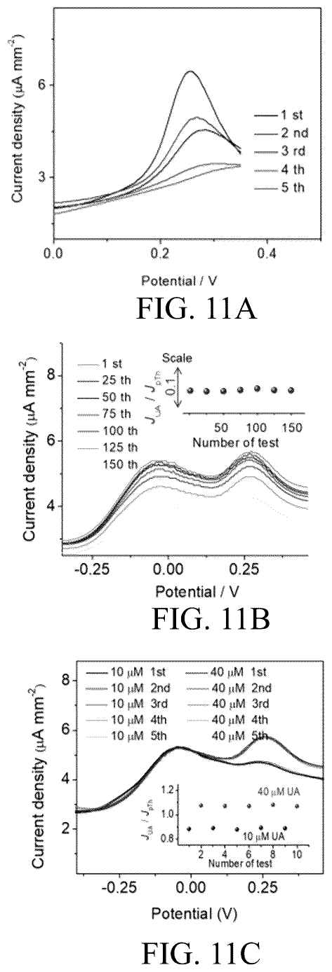

The performances of the fabricated pTh-modified nano Au electrode in accordance with the present invention are characterized in terms of resistance to by-product deposition and avoidance of interference from multi-dimensional disturbances. In contrast to the gradual decay of the response signals of the same UA sample on the conventional nano Au electrode as the number of tests increased (FIG. 11A), the pTh-modified nano Au electrodes could perform dozens of times without substantial difference and signal shift (FIG. 11B), which is mainly attributed to adsorption resistance of pTh. As displayed in the insert of FIG. 11B, as the DPV test cycles increases, it inevitably leads to the synchronous decline of the built-in reference signals (J_{pTh}) and the UA oxidation response signals (J_{UA}), due to the attenuated electrode performance. Obviously, using the ratio evaluation mode (J_{UA}/J_{pTh}) (i.e., ratiometric sensing strategy) in UA measurement can largely repel above potential impact and improve the accuracy for multiple cycle tests.

With the qualification that J_{pTh} and J_{UA} remains above 70% of the original signal, the pTh-modified nano Au electrode withstood 150 consecutive uninterrupted and accurate tests. Subsequently, the repeatability of the UA sensors is further investigated. As shown in FIG. 11C, the low UA concentration (at 10 μM) output results are not interference by high UA concentration (at 40 μM) detection. Its excellent robustness and accuracy greatly guarantee the reusability required for 24/7 long-term monitoring.

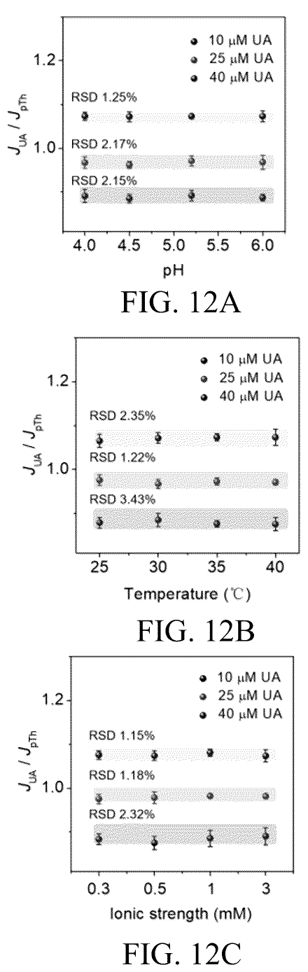

The advantages of the ratiometric sensing strategy of the UA sensors provided by the present invention in shielding the interference from external crosstalks are also studied. FIGS. 12A to 12C described the sensor outputs in response on varied pH, temperature, and ionic strength, respectively. These results suggested that the provided UA sensors are ultra-stable against external environmental factors.

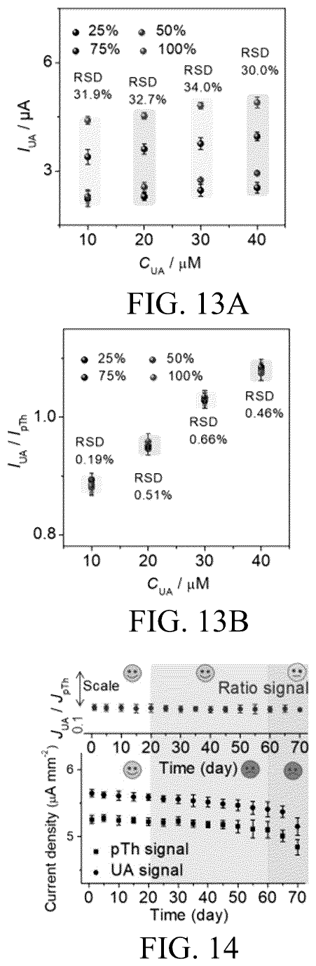

Besides, the effect of extreme condition changes such as electrode breakage raising from long-term sweat immersion or external force on the stability of the output results is examined (FIGS. 13A and 13B). It is calculated that the relative standard deviation (RSD) of the ratiometric means (the ratio of current signals of UA and pTh, I_{UA}/I_{pTh}) is reduced by at least 50 times compared to that of UA signal alone (the current signals of UA alone, I_{UA}), encountering various degrees of electrode losses.

Furthermore, long-term reliability of the provided ratiometric UA sensors after being activated is investigated (FIG. 14). The fabricated UA sensors with such distinguished performance extended the operation lifetime of wearable biosensors and improved the robustness of long-term continuous monitoring, which solved the main bottleneck of wearable biosensors for the long-term practical applications.

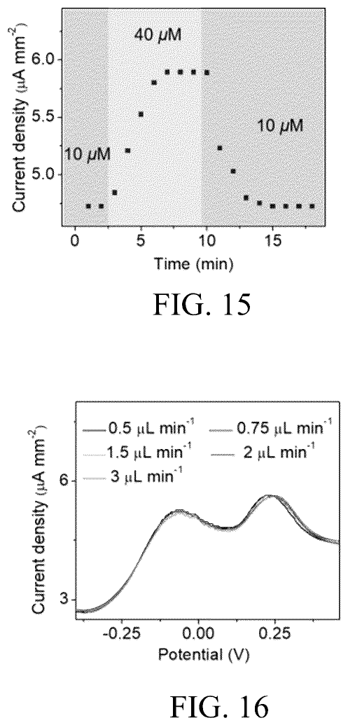

As illustrated in FIG. 15, when the high concentration UA sample (40 μM) is pumped into the microchannel network filled with low concentration sample (10 μM), the real-time current density of the recorded oxidation peak of UA is constantly updated with a 30-s DPV scan every minute, and a new stable signal would be obtained within 4 min. Meanwhile, considering the influence of different sweating rates caused by individual differences, DPV response curves are recorded with the wearable biosensing patch at the physiological sweat rates ranging from 0.5 to 3 μL min⁻¹. As illustrated in FIG. 16, the wearable sensing patch showed very stable reading for the same sample at various flow rates.

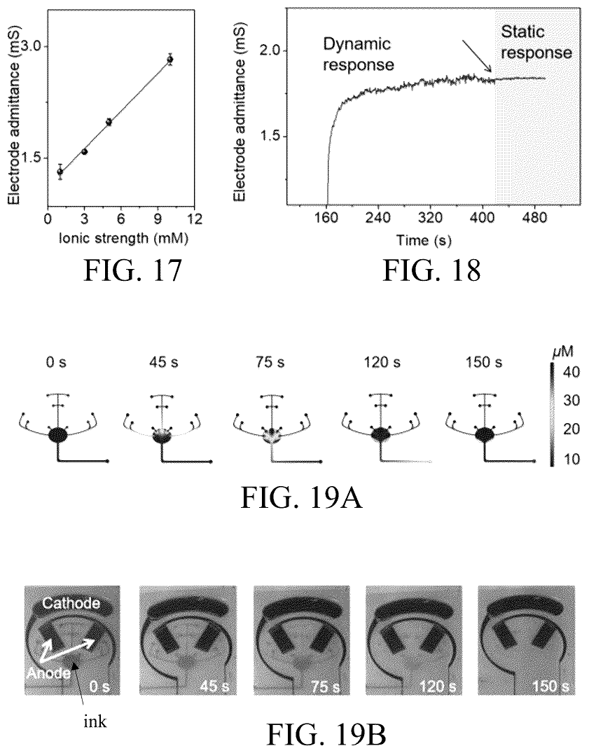

The relevant experimental results indicated that when sweat reaches the admittance analysis electrodes, the admittance pulse undergoes several orders of magnitude transitions from nS to mS, which can be used as an indication for collecting sufficient sweat (FIG. 17). By recording the different responses of electrode admittance to dynamic and static samples (FIG. 18), it is demonstrated that the magnitude of fluctuations in admittance values can be used to unambiguously reflect whether perspiration being sustained.

With the optimized design of the microchannel network (ten inlets, 180-degree span, and aligned to outlet) and 1.0 μL min⁻¹ as the inlet sweat rate, the simulated refreshing time is around 150 s for a sample concentration change of 10 μM to 40 μM (FIG. 19A). During the on-body trials, sweat can be readily induced locally and sampled with high temporal resolution, where the pre-injected ink is expelled by the resulting sweat along microchannels out of the sensing area (FIG. 19B).

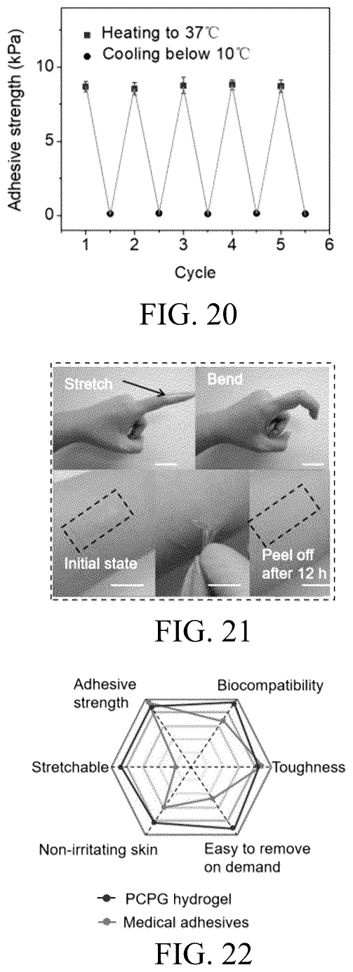

FIG. 20 shows performance of the PCPG hydrogel. When temperature is dropped to 10° C., the boundary of PCPG hydrogel is clear and its adhesion strength almost disappeared, whereas its adhesion strength increased significantly when held at 37° C. for several minutes. After optimizing the proportion of the components, the resultant PCPG hydrogel is soft enough to effectively adhere to knuckle (FIG. 21). The PCPG hydrogel not only ensured long-term wearability requirements, but also exhibited superiorities over medical adhesive tapes in terms of stretchability, biocompatibility, and painless removal (FIG. 22).

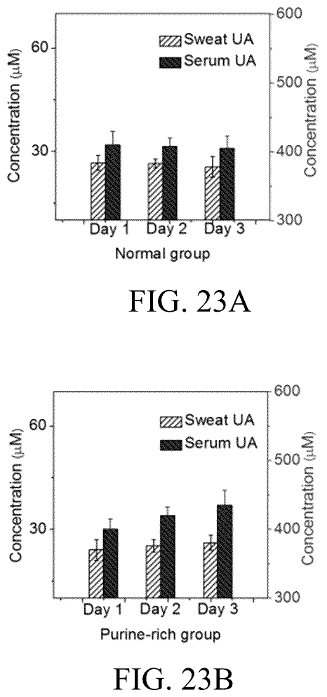

To evaluate the feasibility of the biosensing system provided by the present invention in practical applications, a controlled purine-diet study is conducted in two groups of healthy individuals: normal group (n=3) and purine-rich group (n=3). Both serum and sweat UA levels increased for subjects in purine-rich group, while the readings from normal group did not fluctuate significantly during the tests without additional purine-rich intake (FIGS. 23A and 23B), which is consistent with previously reported results.

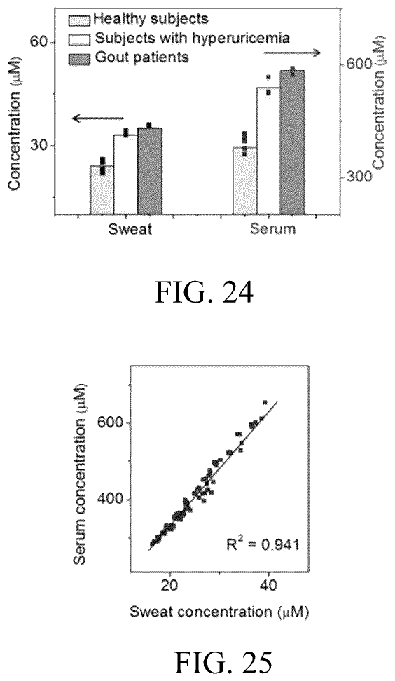

Furthermore, the biosensing system could definitively screen out abnormal individuals (subjects with HUA, n=3 and gout patients, n=2) among healthy individuals (n=5) (FIG. 24). Finally, a correlation between sweat and serum UA levels is established by collecting data from 80 independent human samples, with a correlation coefficient as high as 0.941 (FIG. 25), fully proving the reliability of the biosensing system in reflecting serum UA concentrations.

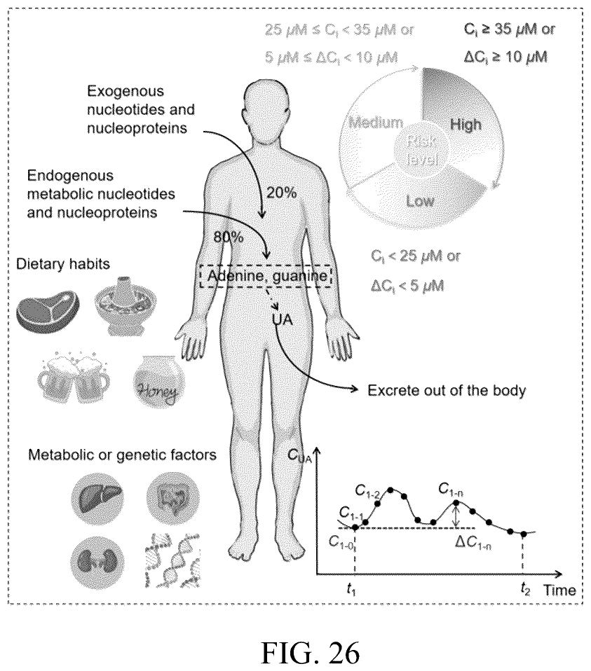

Continuous monitoring UA levels allows early detection of asymptomatic UA metabolic risk, that is significant for effective personalized risk alerts, dietary interventions, and UA-lowering therapy (FIG. 26). Specifically, the biosensing system focuses on real-time UA value (C₁₋₀, C₁₋₁, C₁₋ₙ, . . . , C₂₋₀, . . . ) risk as well as the risk of UA changes (ΔC₁₋₁, ΔC₁₋ₙ, . . . ΔC₂₋ₙ, . . . ) by recording UA levels throughout the day.

Based on the previously established correlation of the UA values in serum and sweat, for real-time UA values, Cᵢ<25 μM (equivalent to serum UA<420 μM) is set as the low risk level; 25 μM≤Cᵢ<35 μM (equivalent to 420 μM≤serum UA<540 μM) is set as the medium risk level, C(M); and Cᵢ≥35 μM (equivalent to serum UA≥540 μM), C(H), is set as the high risk determination criterion. For the evaluation of fluctuating UA values, ΔCᵢ<5 μM (equivalent to fluctuations of serum UA<60 μM) is set as the low risk level; 5 μM≤ΔCᵢ<10 μM (equivalent to 60 μM≤fluctuations of serum UA<120 μM) is set as the medium risk level, ΔC(M); and ΔCᵢ≥10 μM corresponded to fluctuations of serum UA≥120 μM, which is set as the high risk level, ΔC(H). Elevated levels of circulating UA and their dramatic fluctuations should be highly alarming.

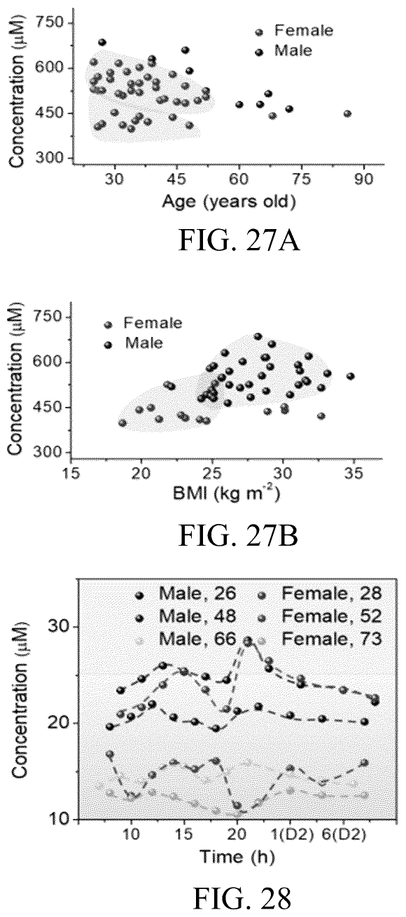

As seen by collecting personal information from 50 randomized patients with HUA, the morbid population shared some common traits, such as more males than females, a predominance of middle-aged and young adults, and a high BMI (FIGS. 27A and 27B).

A pilot study has been carried out using simple control variables to investigate the correlation between UA levels and age throughout the day involving three groups of subjects: youth (25≤age<40), middle-aged (40≤age<60), and elderly (age≥60). Participants in the elderly group had lower UA levels and slower fluctuations throughout the day compared to the middle-aged and younger groups, which may be related to their dietary habits and lower food intake (FIG. 28).

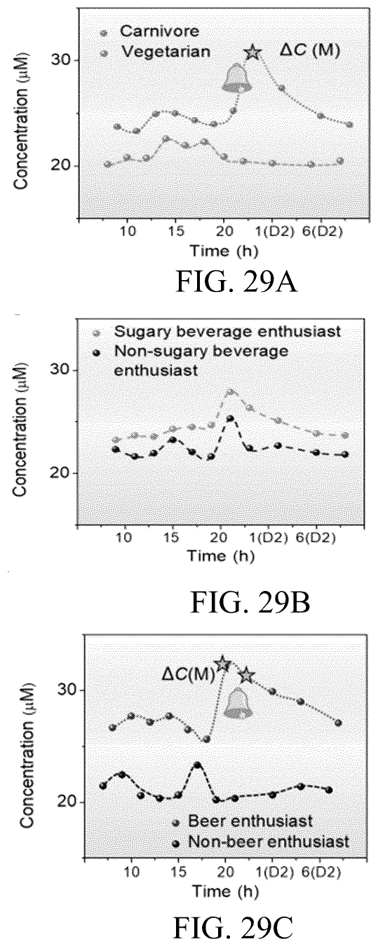

At the same time, UA levels are generally slightly higher in male than in female in the same age group. Additionally, FIGS. 29A to 29C indicated that high intake of meat and beer leads to a substantial increase in UA levels over a period of time, which can be attributed to the high purines production and impaired metabolism of UA.

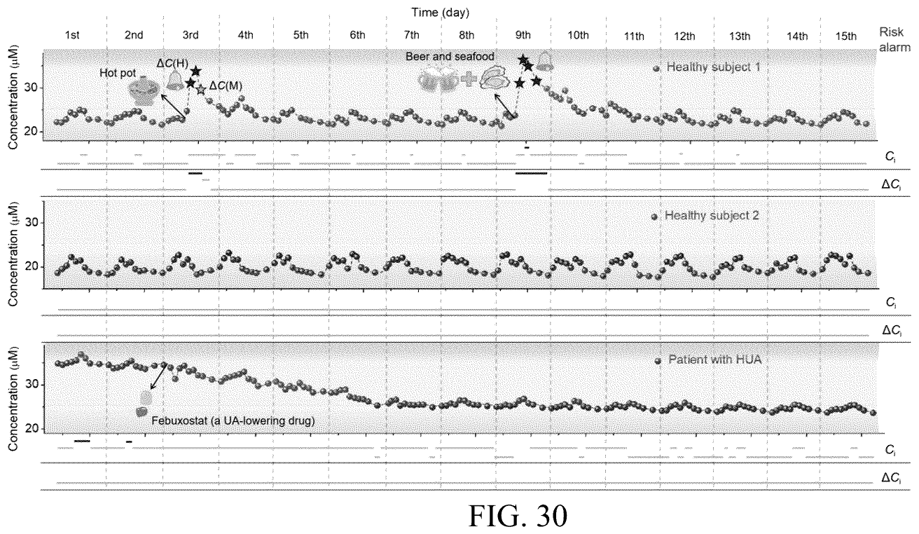

To further evaluate the potential applications of the biosensing system in providing long-term UA monitoring, risk warning and guidance for diet and drug administration, a 15-day monitoring of UA levels in sweat has been conducted on two healthy individuals as well as one HUA patient (FIG. 30). According to the results from healthy subject 1, intake of a high-purine diet not only affected the circulating UA level, but also caused dramatic change over a short period of time. It is worth noting that excessive purine intake is likely to cause the next day and even the third day's UA levels increase, which is due to the excess UA produced requires more time to be metabolized and excreted from body. Meanwhile, UA levels in healthy subject 2 showed regular and flatter fluctuations over a 15-day period under normal or low-purine diets. Furthermore, HUAMS is also employed to track fluctuations in sweat UA levels over a 15-day period in two patients with HUA. The above results fully validated the promise of biosensing system for personalized dosage adjustment in UA-lowering therapy.

While the present disclosure has been described and illustrated with reference to specific embodiments thereof, these descriptions and illustrations are not limiting. The illustrations may not necessarily be drawn to scale. There may be distinctions between the illustrations in the present disclosure and the actual apparatus due to manufacturing processes and tolerances. There may be other embodiments of the present disclosure which are not specifically illustrated. Modifications may be made to adapt a particular situation, material, composition of matter, method, or process to the objective and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations.

What is claimed is:

1. A reusable wearable uric acid (UA) biosensing system comprising:
a flexible printed circuit board; and
a UA sensing patch attached on the flexible printed circuit board and including:
an adhesive interface for on-demand removable adhesion to a skin region;
an iontophoresis interface for facilitating stimulation and control of sweating of the skin region;
a microfluid channel layer having a microchannel network with a plurality of microchannel inlets and a microchannel outlet for collecting sweat from the skin region; and
an electrode layer including respective electrodes for sweat stimulation and UA concentration monitoring.

2. The reusable wearable uric acid (UA) biosensing system of claim 1, wherein the electrode layer includes UA sensing electrodes for measuring UA concentration in the sweat of the skin region and the UA sensing electrodes having a metal/polymer functionalized electrode structure and configured to be operated under a ratiometric sensing mode.

3. The reusable wearable uric acid (UA) biosensing system of claim 2, wherein the metal/polymer functionalized electrode structure is constructed with a metallic thin film deposited with nano metallic particles and coated with a layer of electroactive polymer.

4. The reusable wearable uric acid (UA) biosensing system of claim 3, wherein the metallic thin film and the nano metallic particles are made of gold and the electroactive polymer is a polythionine.

5. The reusable wearable uric acid (UA) biosensing system of claim 4, wherein the UA sensing electrodes are arranged in a three-electrode configuration including a working electrode (WE), a counter electrode (CE) and a reference electrode (RE) for providing a stable potential against which the potential of the working electrode is measured.

6. The reusable wearable uric acid (UA) biosensing system of claim 1, wherein the electrode layer further includes a pair of sweat induction electrodes for applying stimulation current on the skin region to induce sweating through iontophoresis effect.

7. The reusable wearable uric acid (UA) biosensing system of claim 6, wherein the iontophoresis interface is patterned to match with the sweat induction electrodes and positioned to form an iontophoresis medium between the skin and the sweat induction electrodes.

8. The reusable wearable uric acid (UA) biosensing system of claim 7, wherein the iontophoresis interface includes a cathode interfacing medium having a shape matched with the cathode of the sweat induction electrodes; and a pair of anode interfacing mediums each having a shape matched with each anode pad of the sweat induction electrodes.

9. The reusable wearable uric acid (UA) biosensing system of claim 8, wherein the cathode interfacing medium is made of a NaCl hydrogel; and the anode interfacing mediums is made of a carbachol hydrogel.

10. The reusable wearable uric acid (UA) biosensing system of claim 1, wherein the electrode layer further includes a pair of admittance analysis electrodes arranged at opposite side walls of a microchannel outlet of the microchannel network respectively such that the admittance analysis electrodes and the sweat flowing through the microchannel outlet form an electrode-electrolyte interface for quantifying sweating of the skin region.

11. The reusable wearable uric acid (UA) biosensing system of claim 1, wherein the plurality of microchannel inlets distributed over a central area of the skin region; and the microchannel outlet positioned in a peripheral area of the skin region.

12. The reusable wearable uric acid (UA) biosensing system of claim 11, wherein the plurality of microchannel inlets is evenly arranged around two opposite sides of an anode of the sweat induction electrodes in a wrap-around manner.

13. The reusable wearable uric acid (UA) biosensing system of claim 1, wherein the adhesive interface is patterned to have respective openings matched with shapes of iontophoresis interface and the plurality of microchannel inlets.

14. The reusable wearable uric acid (UA) biosensing system of claim 13, wherein the respective openings include a first opening having a shape matched with the cathode interfacing medium; a pair of second openings having shapes matched with the anode interfacing mediums; and a plurality of third openings having shapes matched with the microchannel inlets.

15. The reusable wearable uric acid (UA) biosensing system of claim 14, wherein the adhesive interface is made of a temperature-responsive adhesive hydrogel which forms a soft conformal contact with the skin and adapt with a variety of mechanical deformations.

16. The reusable wearable uric acid (UA) biosensing system of claim 15, wherein the temperature responsive adhesive hydrogel is a PCPG hydrogel.

17. The reusable wearable uric acid (UA) biosensing system of claim 1, wherein the channel layer is made of a layer of polydimethylsiloxane patterned to form the microchannel network.

18. The reusable wearable uric acid (UA) biosensing system of claim 1, wherein the microchannel network has a plurality of microchannels, each sprayed with a layer of monodisperesed silica micropheres to improve surface hydrophilicity.
